(19) United States
(12) Patent Application Publication
(10) Pub. No.: US 2018/0104157 A1
(43) Pub. Date: Apr. 19, 2018

(54) DELIVERING NON-PATHOGENIC, INTESTINALLY BENEFICIAL BACTERIA TO A PATIENT HAVING A FEEDING TUBE

(71) Applicant: Evolve BioSystems, Inc., Davis, CA (US)

(72) Inventors: David KYLE, Davis, CA (US); David MILLS, Davis, CA (US); Samara FREEMAN-SHARKEY, Davis, CA (US)

(21) Appl. No.: 15/567,250

(22) PCT Filed: Apr. 15, 2016
(86) PCT No.: PCT/US16/27932

§ 371 Date: Oct. 17, 2017

Related U.S. Application Data
(60) Provisional application No. 62/149,310, filed on Apr. 17, 2015.

(51) Int. Cl. A61J 15/00; A61K 35/745; A61K 35/747

(52) U.S. Cl. CPC A61J 15/0003; A23L 33/135; A61K 35/747; A61K 35/745

(57) ABSTRACT

Devices and methods for delivering non-pathogenic, intestinally beneficial bacteria to a patient having a feeding tube. Examples of non-pathogenic, intestinally beneficial bacteria that could be used include Bifidobacterium species and Lactobacillus species. In one embodiment, the bacteria is contained in a bacteria infusion device that comprises: (a) an internal chamber; (b) an outlet in communication with the internal chamber; and (c) the non-pathogenic, intestinally beneficial bacteria contained in the internal chamber. Another embodiment is a feeding tube comprising a flexible tubing and coating on the outer surface of the flexible tubing. The coating comprises an antimicrobial agent. Also disclosed are methods of administering a non-pathogenic, intestinally beneficial bacteria to a patient.

### TECHNICAL FIELD

The present invention relates to the use of feeding tubes for delivering nutrition to a patient. In particular, this invention reduces the risk of pathogenic bacteria colonizing the patient by providing antimicrobial treatment of the feeding tube and facilitating introduction of beneficial intestinal bacteria to compete for colonization loci with pathogenic bacteria.

### BACKGROUND

In newborn human infants, protection of the intestinal mucosa may be facilitated by certain bifidobacteria, which prevent binding of certain pathogenic bacteria to mucosal cells by competitive exclusion. Specifically, there is an inverse relationship between the presence of one specific Bifidobacterium, B. longum subspecies infantis (B. infantis), and pathogenic Enterobacteriaceae in full-term infants and in premature infants fed B. infantis. Moreover, B. infantis accelerates the closure of gap junctions between mucosal cells (i.e., accelerating gut maturation) and facilitates the development of the infant's naïve immune system.

These bifidobacteria may be found in the mother's GI tract. Infants are typically inoculated with these bifidobacteria through vaginal delivery. See Le Huerou-Luron et al., “Breast v. formula-feeding: impacts on the digestive tract and immediate and long-term health effects,” Nutr Res Rev. 23:23-36 (2010); Conroy et al., “The long-term health effects of neonatal microbial flora,” Curr Opin Allergy Clin Immunol. 9:197-201 (2009). However, babies in the Neonatal Intensive Care Unit (NICU) are commonly born by cesarean section, which does not expose them to bifidobacteria inoculation provided by vaginal delivery. These babies are also generally fed commercial preterm infant formulas that do not include human milk oligosaccharides which would facilitate the growth of these bifidobacteria (even if the babies had been vaginally delivered and inoculated thereby). Moreover, babies in the NICU are far more likely to be given a treatment of antibiotics. Although the antibiotics are directed to pathogenic bacteria, it will also eliminate most of the beneficial bacteria in the GI tract microbiome as well. As a consequence, most infants in the NICU are dysbiotic for some reason or another.

Newborn infants born prematurely or with certain gastrointestinal (GI) pathologies commonly require enteral feeding from an external nutritional source directly to the stomach of a low birth-weight infant through nasal-gastric (NG) tubes, which can often remain in the stomach for weeks. During this time, there can be a significant build-up of microbial growth on the NG tube. If there is growth of pathogenic bacteria on the NG tube, it can become a significant focus of infection. Overgrowth of pathogenic bacteria can lead to necrotizing enterocolitis with gut necrosis. Necrotizing enterocolitis is a gut necrosis that may be due to maldevelopment of the GI mucosa or gut dysbiosis, leading to an overgrowth of pathogenic bacteria in the gut of the premature infant and often resulting in death or disablement of that newborn infant.

NG-fed babies have at least two issues that may lead to a higher rate of GI problems, such as necrotizing enterocolitis. First, the adventitious microbial growth on the NG tubes needs to be reduced as it represents a significant focus for infection. Second, the babies need to be supplemented with the beneficial bifidobacteria in order to protect the intestinal mucosa from damage and invasion by pathogenic bacteria. This invention provides a solution to both of these contrasting goals in a single device.

### SUMMARY

Various embodiments of the invention include devices and/or methods for reducing adventitious microbial growth on nasal-gastric tubes and protecting intestinal mucosa in a human from damage and invasion by pathogenic bacteria. The device may include a tube with an outer surface, the tube being configured to deliver enteral feeds to the human and a coating including an antimicrobial agent applied to the outer surface. In some embodiments, the enteral feeds may include a beneficial bacteria selected from the group consisting of Bifidobacterium and Lactobacillus.

In various embodiments, the present invention uses non-pathogenic, intestinally beneficial bacteria. Such bacteria may be useful for protecting the intestinal mucosa from invasion by pathogenic bacteria. In some embodiments, the non-pathogenic, intestinally beneficial bacteria may be a Bifidobacterium species or Lactobacillus species. Examples of Bifidobacterium species include B. breve, B. longum (including subspecies infantis), or B. pseudocatenulatum. Examples of Lactobacillus species include L. plantarum, L. pentosus, L. salivarius, L. crispatus, L. sakei, L. antri, or L. coleohominis. In some embodiments, the bacteria used in the present invention is pure, i.e., bacteria of only one species. In some embodiments, there are two or more different types of non-pathogenic, intestinally beneficial bacteria. In some embodiments, the bacteria used in the present invention is characterized as having surface structures (e.g. receptors) that internalize milk glycans or derivative monomers of milk glycans (e.g. fucose, sialic acid, and N-acetylglucosamine). Such bacteria are described in Smilowitz et al., “Breast Milk Oligosaccharides: Structure-Function Relationships in the Neonate” (2014) Annu Rev Nutr , vol 34:143-169; and in International Patent Application Ser. No. PCT/US15/57226 and Ser. No. PCT/US16/22226, all of which are incorporated by reference herein in their entirety.

An embodiment of the present invention provides a bacteria infusion device that comprises: (a) an internal chamber; (b) an outlet in communication with the internal chamber; and (c) a non-pathogenic, intestinally beneficial bacteria contained in the internal chamber. The device may contain any suitable amount of the non-pathogenic, intestinally beneficial bacteria. In some embodiments, the amount of the bacteria is 0.1 billion to 100 billion live colony forming units (cfu); in some cases, 1 billion to 50 billion cfu; and in some cases, 5 billion to 20 billion cfu.

The bacteria may be provided in any suitable physical form, such as liquid, solid, or semi-solid (e.g. a gel). In some embodiments, the bacteria is provided in a solid form; in some embodiments, the bacteria is provided in a liquid medium. In some embodiments, a bacteria-preserving agent is mixed with the bacteria. Examples of bacteria-preserving agents include trehalose, sorbitol, albumin, polyethylene glycol, polyvinylpyrrolidone (PVP), milk fractions, dimethyl sulfoxide, and glycerol.

Any suitable volume of the liquid medium can be used. In some embodiments, the volume of liquid medium is in the range of 0.3 ml to 5 ml. For example, the volume of the liquid medium can be about 0.5 ml, about 1 ml, about 1.5 ml, about 2 ml, about 2.5 ml, about 3 ml, about 3.5 ml, about 4 ml, about 4.5 ml, or about 5 ml.

In embodiments where the bacteria is in a liquid medium, in some cases, the liquid medium comprises a nutritional ingredient, such as an oil, a mammalian milk, or an artificial nutritional formulation. An example of a nutritional oil is a triglyceride oil, such as medium chain triglyceride oils (having aliphatic tails of 10-14 carbon atoms). It is not necessary that the liquid medium comprise any nutritional ingredients. For example, the liquid medium may comprise mineral oil.

In some embodiments, the bacteria infusion device further comprises an outlet fitting for coupling the outlet to a feeding tube. In some embodiments, the bacteria infusion device further comprises an inlet and an inlet fitting for coupling the inlet to an enteral feed source. The inlet and/or outlet fittings may have any suitable configuration. In some embodiments, the inlet fitting, outlet fitting, or both are standardized medical connection fittings. In some embodiments, the medical connection fitting comprises a threaded lock (e.g., a luer lock). Other types of fittings that could be used include stepped spike connectors, barbed locks, quick connect fittings, or Swagelok fittings. In some embodiments, the inlet fitting, outlet fitting, or both are configured to be incompatible with a standard luer lock. Since luer locks are commonly used in intravenous lines, having an incompatible fitting can be useful for reducing the risk of accidental misconnection of the enteral feed to an intravenous line.

The device may have any suitable dimensions. In some embodiments, the device has a central diameter in the range of 0.5 cm to 4 cm. In some embodiments, the device has a length in the range of 1 cm to 10 cm.

In some embodiments, the internal chamber is oxygen-free. The absence of oxygen could be useful for maintaining the viability of the intestinally beneficial bacteria, especially for bacteria that are obligate anaerobes.

In another embodiment, the present invention provides a feeding tube that comprises a flexible tube. The feeding tube comprises a proximal end and a distal end. The feeding tube may have any suitable configuration as a gastrointestinal feeding tube. For example, the feeding tube may be configured as a nasal-gastric feeding tube. Other types of gastrointestinal feeding tubes include percutaneous feeding tubes, such as gastrostomy tubes or gastrojejunostomy tubes.

The feeding tube may be made of any suitable material. For example, the feeding tube may comprise polyurethane, silicone, polyvinyl chloride, polyethylene, or thermoplastic polymer. The feeding tube may have one or more lumens. For example, the feeding tube may be a dual lumen feeding tube (e.g., one lumen for designated for feeds and another lumen designated for medications)

In some embodiments, the feeding tube further comprises a bacteria infusion device of the present invention that is coupled its proximal end. The feeding tube may comprise a feed port for connection with the enteral feed source and/or the bacteria infusion device. The feed port may comprise any suitable type of fitting, including any of the standardized medical connection fittings as described above.

In another embodiment, the present invention provides a kit that comprises a feeding tube and a bacteria infusion device of the present invention. In some embodiments, the feeding tube comprises a feed port and the bacteria infusion device comprises an outlet fitting that is compatible with the feed port of the feeding tube.

In another embodiment, the present invention is a method of assembling a kit of the present invention. The bacteria infusion device has a proximal end and a distal end. The method comprises coupling the distal end of the bacteria infusion device to the feeding tube. In some embodiments, the method further comprise coupling the proximal end of the bacteria infusion device to an enteral feed source.

In various embodiments, the present invention provides a feeding tube comprisinga flexible tubing having an outer surface and a coating on the outer surface, where the coating comprises an antimicrobial agent. Any suitable type of antimicrobial agent may be used. In some embodiments, the antimicrobial agent is a protein or peptide. In some embodiments, the antimicrobial agent is a milk protein; examples of milk proteins that could be used include lactoferrin or lysozyme. In some embodiments, the antimicrobial agent is a naturally-occurring compound; examples of such antimicrobial agents include allicin, catechin, citricidal, cinnamaldehyde, and carvacrol. Other types of antimicrobial agents that could be used include zinc salts, silver salts, flavanones, or synthetic compounds.

The coating may be applied to the flexible tubing using any suitable method. In some embodiments, the antimicrobial agent is covalently immobilized on the outer surface. In some embodiments, the antimicrobial agent is covalently immobilized on the outer surface through a linker moiety. One technique that could be used to covalently bond the antimicrobial agent to the outer surface is the diazotization method described in U.S. Pat. No. 3,574,062, the entire disclosure of which is incorporated herein by reference. Other techniques that could be used include those described in International Patent Application Publication No. WO 2001/056627; U.S. Pat. No. 7,976,863; Kim et al., “Protein immobilization techniques for microfluidic assays” (2013 July) Biomicrofluidics, 7(4): 041501; and Mohamad et al., “An overview of technologies for immobilization of enzymes and surface analysis techniques for immobilized enzymes” (2015 February) Biotechnology & Biotechnological Equipment, 29(2):205-220. The entire disclosures of all the preceding are incorporated by reference herein.

In embodiments where the feeding tube comprises a bacteria infusion device of the present invention as described herein, the feeding tube may be coated with an antimicrobial agent as described herein. The feeding tube may comprise a feed port for connection with an enteral feed source and/or the bacteria infusion device. The feed port may comprise any suitable type of fitting, including any of the standardized medical connection fittings as described above.

Some embodiments of the present invention provide a method of administering a non-pathogenic, intestinally beneficial bacteria to a patient. The patient has a feeding tube inserted into the gastrointestinal tract. The method comprises infusing the non-pathogenic, intestinally beneficial bacteria into the patient's gastrointestinal tract (e.g., stomach, duodenum, or jejunum) through the feeding tube.

The bacteria may be provided in any suitable physical form as explained above. In some embodiments, the bacteria is provide in a liquid medium. The volume of liquid medium may be as described above. In some embodiments, the method further comprises, prior to use, storing the bacteria at a temperature that is below 20° C. (e.g., in a refrigerator).

The non-pathogenic, intestinally beneficial bacteria could be administered repeatedly to the patient. In some embodiments, the method further comprises repeating the step of infusing the bacteria into the patient's gastrointestinal tract. In some embodiments, a subsequent infusion of bacteria is performed within 12 hours to 72 hours after the initial infusion (e.g., daily administration).

The method could be implemented using a bacteria infusion device of the present invention. In some embodiments, the method comprises coupling the bacterial infusion device to the feeding tube, the bacteria being infused out from the internal chamber through the outlet of the bacteria infusion device.

In some embodiments, the method further comprises coupling an enteral feed source to the bacteria infusion device. In some embodiments, the method further comprises infusing the enteral feed source through the bacteria infusion device and into the feeding tube. In some embodiments, the bacteria is infused simultaneously with infusion of the enteral feed. In some embodiments, the method further comprises detaching the bacteria infusion device after the enteral feed infusion is complete.

Any suitable type of feeding tube could be used for implementing the method. In some embodiments, the feeding tube is an antimicrobial-coated feeding tube of the present invention.

The present invention could be used in any mammal, including humans. The present invention could be used in a variety of different types of patients, including adult patients and pediatric patients. Examples of pediatric patients include infants, such as preterm infants.

### BRIEF DESCRIPTION OF THE DRAWINGS

FIGS. 1A and 1B show an example of a bacterial infusion device of the present invention. FIG. 1A shows a cross-section side view; FIG. 1B shows a perspective view.

FIGS. 2A and 2B show another example of a bacterial infusion device of the present invention. FIG. 2A shows a cross-section side view; FIG. 2B shows a perspective view.

FIG. 3 shows an example of a feeding tube of the present invention.

FIG. 4 shows the bacterial infusion device of FIGS. 2A and 2B attached to the feeding tube of FIG. 3.

FIGS. 5A and 5B show a feeding tube inserted into an infant. FIG. 5A shows a cross-section side view; FIG. 5B shows a front view.

### DETAILED DESCRIPTION OF THE EMBODIMENTS

The device and method of the present disclosure may be used to reduce the adventitious microbial growth on NG tubes and protect the intestinal mucosa from damage and invasion by pathogenic bacteria and may be comprised of the following elements: a tube with an antibacterial agent coating. This list of constituent elements is intended to be exemplary only, and it is not intended that this list be used to limit the device and method of the present application to just these elements. Persons having ordinary skill in the art relevant to the present disclosure may understand there to be equivalent elements that may be substituted within the present disclosure without changing the overall invention.

By way of example, some embodiments of the invention include a method and device for reducing the adventitious microbial growth on NG tubes and protecting the intestinal mucosa from damage and invasion by pathogenic bacteria, particularly in a preterm infant, the device comprising a tube with an outer surface and a coating comprising an antibacterial agent applied to the outer surface. In some embodiments, the antibacterial agent may comprise at least one protein, such as a milk protein, such as lactoferrin or lysozyme. Alternatively or additionally, the antibacterial agent may comprise a natural antibacterial compound, such as allicin, catechin, citricidal, cinnamaldehyde, and carvacrol. The coating may also comprise non-natural antibacterial compounds, such as zinc salts, silver salts, flavanones, and commercial antibiotics. The coating may be applied to the tube using any suitable method and, in some embodiments, the coating's composition may be covalently bonded to the outer surface, where the covalent bond may include a linker moiety positioned between the tube and the protein. In another alternative, the coating may be a whey protein film which may be applied by solvent casting from a whey protein stock which has been enriched in antibacterial proteins, such as lactoferrin and/or lysozyme.

The tube may be made of any suitable material, such as a material typically used in the enteral feeding of a mammal, such as a human infant that is premature or of low birth weight. For example, the tube may comprise a polyurethane, silicone, polyethylene, polyvinyl chloride, or thermoplastic polymer.

In another embodiment, the tube may be connected to a bacterial infusion device which comprises a pure bacterial culture in the central volume of the device, where the pure bacterial culture may be present at a level of from about 0.1 billion to 100 billion live colony forming units of the pure bacterial culture. The beneficial bacteria may comprise bacteria that consume milk glycans or their derivative monomers, such as fucose, sialic acid, and N-acetylglucosamine. For example, the pure bacterial culture may comprise a culture from the genus Bifidobacterium or Lactobacillus. The Bifidobacterium species may B. breve, B. longum, or B. pseudocatenulatum and, in some embodiments, comprises B. longum subsp. infantis. The Lactobacillus species may be L. plantarum, L. pentosus, L. salivarius, L. crispatus, L. sakei, L. antri, or L. coleohominis. Cultures for use in this invention may be produced by any suitable culture technique known to be effective for the particular organisms. Exemplary protocols are described in International Patent Application Ser. No. PCT/US15/57226 and Ser. No. PCT/US16/22226, both of which are incorporated by reference herein in their entirety.

During fermentation of the organism that is activated to use mammalian milk oligosaccharides, the organism may release high amounts of short chain fatty acids, such as acetate, into the supernatant. These short chain fatty acids may have the beneficial effect of suppressing the growth of other potentially pathogenic organisms. As such, the supernatant that is separated from the organism may be stored and used to flush the line following feeding and/or delivery of the bacterial culture. Alternatively, a cell slurry of the bacterial culture plus its supernatant may be frozen and the combination delivered in the infusion device (sealed chamber) that is warmed to 37° C. before use.

The pure bacterial culture may be present in a formulation that may aid in the preservation of the viability of the pure bacterial culture during storage and delivery. The formulation may comprise, for example, a stabilizing agent, such as trehalose, dimethyl sulfoxide (DMSO), polyethylene glycol (PEG), glycerol, milk fractions, polyvinylpyrrolidone (PVP), sorbitol, or albumin. The formulation may be a dry formulation prepared by, for example, freeze drying. The formulation may also be suspended or dissolved in a liquid such as, but not limited to, an oil, a triglyceride oil, human milk or a fraction thereof, and an artificial nutritional formula prepared for the human in need of the bacteria. Alternatively, the formulation may be semi-solid (e.g., a gel). For stability during storage, it is preferred that the formulation have low water activity (e.g., water activity less than or equal to 0.35). For liquid or semi-solid formulations, in some embodiments, the bacteria-containing formulation may be kept frozen. This may be useful in maintaining the bacteria in a dormant state prior to delivery. Transition from dormant metabolic state to active metabolic state may occur ex vivo or in vivo.

If the transistion occurs ex vivo for frozen samples, the transition would involve a protocol of warming the frozen samples and holding in the sealed internal chamber at 37° C. to “wake” up the organisms before injecting or opening the flow into the infant. In the case of the powders or semi-solid low water activity formulations, this would involve adding an aqueous component and holding it at 37° C.

The device comprising the bacterial culture may have a first end and a second end, each end comprising a locking fitting configured to engage with or be coupled to a NG feeding tube such as described in the first embodiment and an enteral feeding source, such as a luer lock, a threaded lock, a barbed lock, a quick connect fitting, or a Swagelok fitting.

In some embodiments, the invention may comprise a plurality of tubes, such as two tubes, where a first tube is an enteral feeding tube and a second tube is for infusion of a pure bacterial culture. The first and/or second tube may be coated with the antibacterial agent described above.

### DETAILED DESCRIPTION OF THE DRAWINGS

To assist in understanding the present invention, particular embodiments are described in detail with references to the figures. These particular embodiments are presented as illustrative examples only. Moreover, the constituent elements in these particular embodiments are intended to be exemplary only and not intended to limit the present invention. It will be apparent to persons having ordinary skill in the art that the present invention is not limited to the embodiments set forth below and that the present invention can be adapted for any suitable application, limited only by the appended claims.

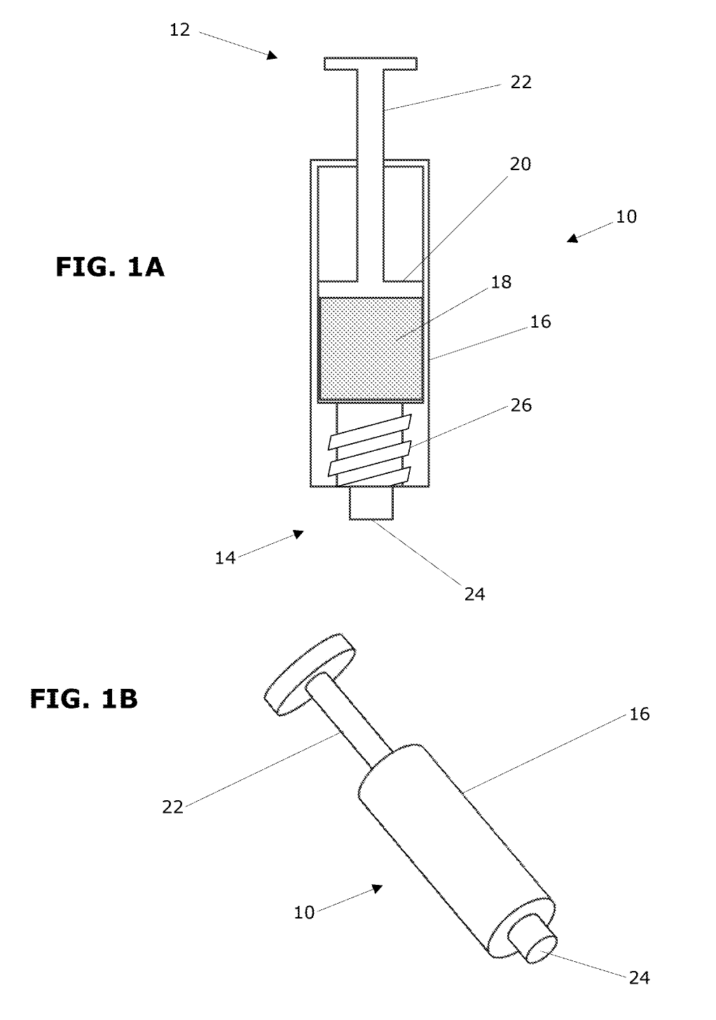

FIGS. 1A and 1B show a syringe-like device 10 having a proximal end 12 and a distal end 14. At its distal end 14, there is an outlet 24. The device 10 has an internal chamber 16 containing 1 mL of a liquid medium 18 of B. infantis bacteria suspended in one gram of medium chain triglyceride oil at a concentration of 10 billion cfu/mL. One end of the internal chamber 16 is sealed with a tightly-fitted piston 20, which is movable within the internal chamber 16 in a proximal-to-distal direction. The piston 20 is connected to a plunger 22 that the user can push to move the piston 20 in a downward direction (with respect to the illustration).

The internal chamber 16 is in fluid communication with the outlet 24. An end cap (not shown) is fitted over outlet 24 at the distal end 14 to seal the bacterial medium 18 inside the internal chamber 16. At the distal end 14 is a threaded fitting 26 for connecting to a feeding tube. In operation, the user removes the distal cap and connects the outlet 24 to a feeding tube via the fitting 26. The user then pushes the plunger 22 to eject the bacterial medium 18 into the feeding tube.

This syringe device 10 can be manufactured in any suitable manner. In one example, a sample of Bifidobacterium longum subsp. infantis is prepared and freeze-dried in the presence of a cryoprotectant (trehalose). Once dry, the material is quantified and diluted to about 100 billion cfu/g using pharmaceutical grade lactose. Approximately 100 mg of this material is then suspended in 1 mL of MCT oil. With the piston 20 pushed all the way down to expel any air inside the syringe device 10, the distal outlet 24 is immersed in the liquid suspension of bacteria and drawn into the internal chamber 16 by drawing back on the plunger 22. Any air drawn into the syringe device 10 is gently expelled. The distal outlet 24 is then capped to seal the bacterial medium inside the syringe device 10. The filled syringe device 10 is stored under sealed and refrigerated conditions until use.

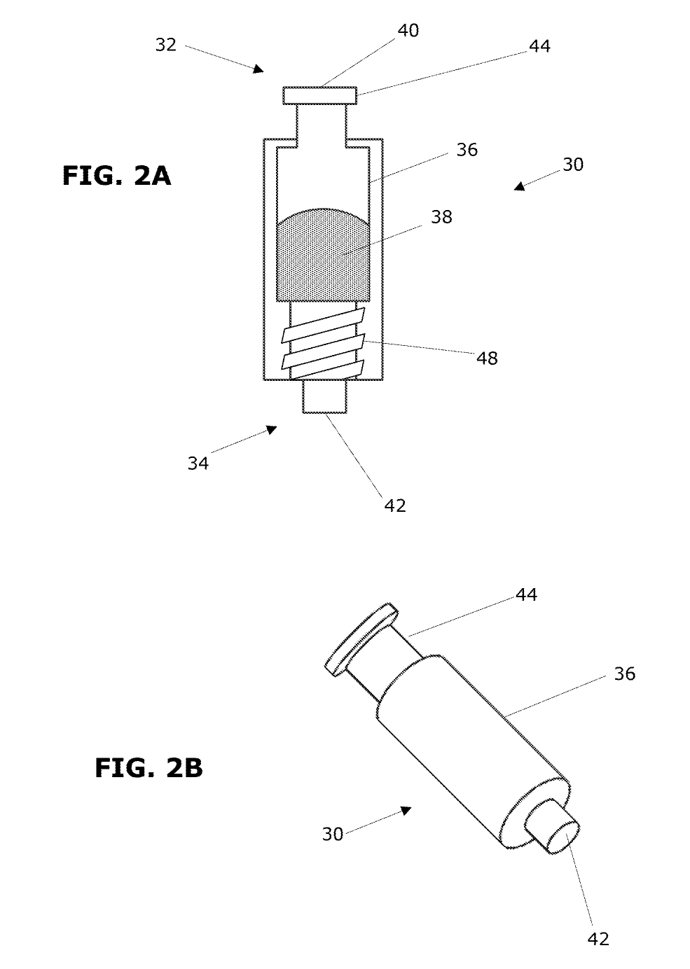

FIGS. 2A and 2B show a dual port, vial-like cassette device 30 having a proximal end 32 and a distal end 34. At its proximal end 32, there is an inlet 40. At its distal end 34, there is an outlet 42. The cassette device 30 has a central diameter of about 1 cm and a length of about 2 cm. The device 30 has an internal chamber 36 containing B. infantis bacteria 38 in powder form. The internal chamber 36 is in fluid communication with the inlet 40 and the outlet 42. Both the inlet 40 and outlet 42 are sealed by removable caps (not shown). At the proximal end 32 of the device 30, there is a threaded inlet fitting 44 for connecting to an enteral feed source. At the distal end 34 of the device 30, there is a threaded outlet fitting 48 for connecting to a feeding tube. The operation of the cassette device 30 is described below in relation to the feeding tube.

This cassette device 30 can be manufactured in any suitable manner. In one example, a sample of Bifidobacterium longum subsp. infantis is prepared and freeze-dried in the presence of a cryoprotectant (trehalose). One end of an empty cassette device 30 is closed with a luer lock cap, and the unit is filled with the freeze-dried B. infantis material. The top of the cassette device 30 is flushed with nitrogen and sealed with a second luer lock cap over outlet 42. The filled cassette device 30 is stored under sealed and refrigerated conditions until use.

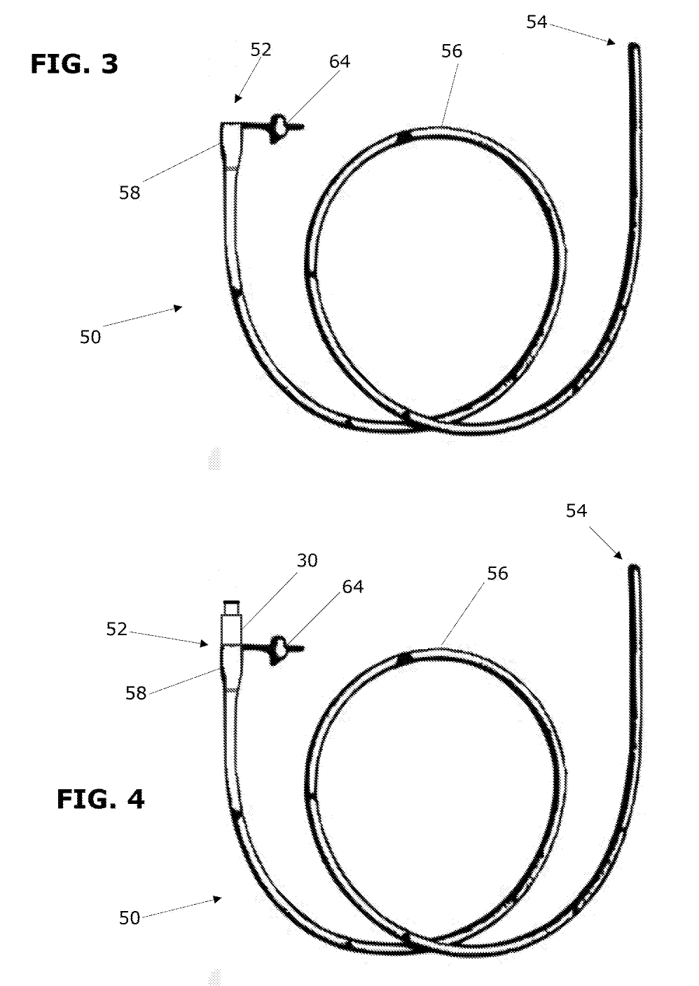

FIG. 3 shows a feeding tube 50 having a proximal end 52, a distal end 54, and a flexible tubing 56. At the proximal end 52 of the feeding tube 50 is a feeding port 58 that ordinarily connects to an enteral feeding source. Attached by a tether is a removable cap 64 for capping feed port 58. Feeding tube 50 may have any suitable length. In some embodiments, the length of the feeding tube is in the range of 30-140 cm.

The outer surface of the flexible tubing 56 of the feeding tube 50 has a coating of lactoferrin as an antimicrobial agent. The lactoferrin is immobilized onto the outer surface by covalent bonding. The lactoferrin could be covalently bonded to the outer surface of the flexible tubing 56 using the method described in U.S. Pat. No. 3,574,062, the entire disclosure of which is incorporated herein by reference. This method could be implemented on a commercially-available polyurethane NG tube that is diazotized by reacting with the diazonium salt of an amino acid (e.g. hippuric acid). An intermediate protein layer is created by coupling bovine serum albumin to the diazo-hippurate by reacting with N,N′-dicyclohexyl carbodiimide. The antibacterial protein is then coupled to the bovine serum albumin layer by reacting with N,N′-dicyclohexyl carbodiimide.

The feeding tube 50 can be manufactured in any suitable manner. In one example, a commercially-available 20 inch polyurethane pediatric NG feeding tube is obtained. The distal end of the NG tube is sealed tightly with a clamp or a plug to prevent any liquid from entering the tube. The distal 16 inches of the NG tube is then coated with lactoferrin in the manner described above. After thorough rinsing and drying of the feeding tube 50, it is re-cut just above the site of the clamp or plug.

FIG. 4 shows the feeding tube 50 combined with the cassette device 30. The feeding tube 50 may be provided with the cassette device 30 attached. Or alternatively, the feeding tube 50 and cassette device 30 may be provided separately, e.g., in a kit.

In operation, the cassette device 30 is taken out from cold storage and warmed to room temperature. The outlet 42 on the cassette device 30 is uncapped and the outlet fitting 48 is connected to the feed port 58 of the feeding tube 50. Next, the inlet 40 on the cassette device 30 is uncapped and the inlet fitting 44 is connected to the enteral feed source. The enteral feed infusion is then started. The enteral feed enters the internal chamber 36 of device 30 through the inlet 40 and mixes with the bacteria powder 38. With this mixing, the bacteria powder 38 is suspended in the liquid enteral feed. With the bacteria mixed therein, the enteral feed continues to travel out through the outlet 42 of the cassette device 30 and into the feeding tube. After the bacterial powder 38 is depleted, the cassette device 30 may be disconnected from the feeding tube and the enteral feed source. Repeated administration of the bacteria (e.g., once daily) could be performed by attaching a new cassette device 30 to the feeding tube 50.

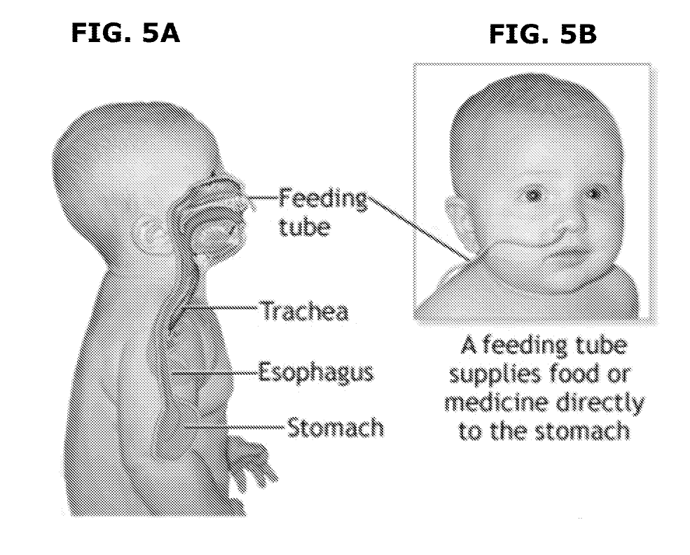

FIGS. 5A and 5B show an NG feeding tube inserted into an infant. The feeding tube is inserted through the infant's nose until the distal end is placed into the stomach. The syringe-like device 10 of FIGS. 1A and 1B could be connected to the proximal end of the NG feeding tube. The B. infantis bacteria carried in the device 10 is ejected into the feeding tube by pushing the plunger 22. Alternatively, the cassette device 30 of FIGS. 2A and 2B could be connected to the proximal end of the NG feeding tube. An enteral feed line could be connected to the proximal end of cassette device 30 and the enteral feeding could be infused through the cassette device 30 into the feeding tube.

### EXAMPLES

### Example 1: Preparation of a Feeding Tube Coated With Lactoferrin

A commercial brand of a 20 inch polyurethane pediatric nasogastric feeding tube with a single or dual port connector is obtained. The distal end of the NG tube is first sealed tightly with a clamp or a plug to prevent any liquid from entering the tube. The distal 16 inches of the NG tube is then diazotized with a diazonium salt of an amino acid by, for example, the method disclosed by U.S. Pat. No. 3,574,062, the entire disclosure of which is incorporated herein by reference, and then reacted with purified lactoferrin or devitalized forms thereof to produce a coating of lactoferrin on the outside surface of the polyurethane tube. After thorough rinsing and drying of the tube, it is re-cut just above the site of the clamp or plug. This or an equivalent tube may be used in any of the kits or methods of this invention.

### Example 2: Use of the Antimicrobial-Coated Feeding Tube

The feeding tube of Example 1 is gently placed through the nose or mouth of an infant and into the stomach using standard practices. In babies with feeding problems, the tip of the tube may be placed past the stomach into the small intestine, providing slower, continuous feedings. The tube is then connected to a supply source of an enteral feed and can be used to provide feedings and medications into the stomach of the baby for many days without significant build-up of bacteria on the feeding tube. The enteral feed may contain mammalian milk oligosaccharides as disclosed in U.S. Pat. No. 8,197,872, International Patent Application Publication No. WO 2012/009315, and International Patent Application Ser. No. PCT/US15/57226, the entire disclosures of which are incorporated by reference herein. Equivalent devices may be used in similar methods according to this invention.

### Example 3: Use of Bacteria Infusion Device with Conventional Feeding Tube

A plastic luer lock device is prepared or obtained from a commercial vendor. The unit has male and female luer lock fittings at either end and a central diameter of about 1 cm and a length of about 2 cm. A sample of Bifidobacterium longum subsp infantis is prepared and freeze-dried in the presence of a cryoprotectant (trehalose). Once dry, the material is quantified and diluted to about 100 billion cfu/g using pharmaceutical grade lactose. Approximately 100 mg of this material is then suspended in 1 mL of MCT oil. One end of the luer lock device is closed with a luer lock cap, and the unit is filled with 1 mL of the MCT oil containing 10 billion cfu of B. infantis. The top of the unit is flushed with nitrogen and sealed with a second luer lock cap. The filled unit is stored under sealed and refrigerated conditions until use. Once a standard feeding tube is inserted into a subject, the filled unit can be removed from storage, warmed to body temperature and connected to the feeding tube between the feeding tube and the source of enteral feeds. The enteral feed may contain mammalian milk oligosaccharides as disclosed in U.S. Pat. No. 8,197,872, International Patent Application Publication No. WO 2012/009315, and International Patent Application Ser. No. PCT/US15/57226, the entire disclosures of which are incorporated by reference herein. Once the enteral feed pump is started, the MCT oil containing the beneficial bacteria is transferred into the stomach of the subject. Equivalent devices may be used in similar methods according to this invention.

### Example 4: Use of the Bacteria Infusion Device and an Antimicrobial Coated Feeding Tube

The bacteria-containing tube of Example 3 is locked into the lactoferrin-coated feeding tube of Example 1 before or after the insertion of the feeding tube into the infant in need of enteral feeding. The bacteria-containing tube is then connected to a supply source of an enteral feed. The enteral feed may contain mammalian milk oligosaccharides as disclosed in U.S. Pat. No. 8,197,872, International Patent Application Publication No. WO 2012/009315, and International Patent Application Ser. No. PCT/US15/57226, the entire disclosures of which are incorporated by reference herein. The first feeding will provide the infant with a first daily dose of B. infantis along with 1 g of MCT oil. After the first feed, the bacteria-containing tube can be removed and subsequent enteral feeds for that day can be provided directly to the infant via the NG tube. Daily additions of the B. infantis can be made with the insertion of a new bacteria-containing tube to the NG tube, and this process can be maintained for as long as the infant is taking enteral feeds. Equivalent devices may be used in similar methods according to this invention.

### Example 5: Feeding Tube Laminated With Whey Protein Film

Another example of an antimicrobial coating that could be used is a whey protein film, such as those described in H. Jooyandeh, “Whey Protein Films and Coatings: A Review,” Pakistan Journal of Nutrition 10(3): 296-301 (2011), the entire disclosure of which is incorporated by reference herein. The whey protein film may have an antimicrobially-effective amount of lactoferrin and/or lysozyme. The whey protein film could be applied by solvent casting or as a laminate sheet around a feeding tube to provide the antimicrobial coating on feeding tubes according to this invention.

The above-described embodiments and examples of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

1. A bacteria infusion device comprising:
an internal chamber;
an outlet in communication with the internal chamber; and
a non-pathogenic, intestinally beneficial bacteria contained in the internal chamber.

2. The device of claim 1, wherein the bacteria is a Bifidobacterium species.

3. The device of claim 1 or 2, wherein the bacteria is B. breve, B. longum, or B. pseudocatenulatum.

4. The device of any one of claims 1-3, wherein the bacteria is B. longum of subspecies infantis.

5. The device of claim 1, wherein the bacteria is a Lactobacillus species

6. The device of claim 1 or 5, wherein the bacteria is L. plantarum, L. pentosus, L. sahvarius, L. crispatus, L. sakei, L. antri, or L. coleohominis.

7. The device of any one of claims 1-6, wherein the amount of bacteria is 0.1 billion to 100 billion live colony forming units.

8. The device of any one of claims 1-7, further comprising a bacteria-preserving agent mixed with the bacteria.

9. The device of claim 8, wherein the bacteria-preserving agent is trehalose, sorbitol, albumin, polyethylene glycol, polyvinylpyrrolidone, milk fractions, dimethyl sulfoxide, or glycerol.

10. The device of any one of claims 1-9, wherein the bacteria is in a solid form.

11. The device of any one of claims 1-9, wherein the bacteria is in a liquid medium.

12. The device of claim 11, wherein the volume of liquid medium is in the range of 0.3 ml to 5 ml.

13. The device of claim 12, wherein the volume of liquid medium is about 0.5 ml, about 1 ml, about 1.5 ml, about 2 ml, about 2.5 ml, about 3 ml, about 3.5 ml, about 4 ml, about 4.5 ml, or about 5 ml.

14. The device of any one of claims 1-13, wherein the device further comprises a liquid medium which comprises a nutritional ingredient.

15. The device of claim 14, wherein the nutritional ingredient is an oil, a mammalian milk, or an artificial nutritional formulation.

16. The device of claim 15, wherein the oil is a triglyceride oil.

17. The device of claim 16, wherein the oil is a medium chain triglyceride oil.

18. The device of claim 15 wherein the nutritional formulation comprises bacterial supernatant.

19. The device of any one of claims 1-18, wherein the bacteria has surface structures that internalize milk glycans or derivative monomers of milk glycans.

20. The device of any one of claims 1-19, further comprising an outlet fitting for coupling the outlet to a feeding tube.

21. The device of any one of claims 1-20, further comprising an inlet and an inlet fitting for coupling the inlet to an enteral feed source.

22. The device of claim 21, wherein the inlet fitting, outlet fitting, or both are standardized medical connection fittings.

23. The device of claim 22, wherein the medical connection fitting comprises a threaded lock.

24. A feeding tube comprising:
a flexible tubing;
a feed port on the flexible tubing; and
a bacteria infusion device of any one of claims 1-23 coupled to the feed port.

25. The feeding tube of claim 24, wherein the feeding tube is a naso-gastric feeding tube.

26. A feeding tube comprising:
a flexible tubing comprising an outer surface; and
a coating on the outer surface of the flexible tubing, the coating comprising an antimicrobial agent.

27. The feeding tube of claim 26, wherein the feeding tube is a naso-gastric feeding tube.

28. The feeding tube of claim 26 or 27, wherein the antimicrobial agent is a protein.

29. The feeding tube of any one of claims 26-28, wherein antimicrobial agent is a milk protein.

30. The feeding tube of any one of claims 26-29, wherein the antimicrobial agent is lactoferrin or lysozyme.

31. The feeding tube of any one of claims 26-30, wherein the antimicrobial agent is a naturally-occurring compound.

32. The feeding tube of claim 31, wherein the antimicrobial agent is allicin, catechin, citricidal, cinnamaldehyde, or carvacrol.

33. The feeding tube of claim 26 or 27, wherein the antimicrobial agent is a synthetic compound.

34. The feeding tube of any one of claims 26-33, wherein the antimicrobial agent is covalently immobilized to the outer surface of the flexible tubing.

35. The feeding tube of any one of claims 26-34, further comprising a bacteria infusion device of any one of claims 1-23.

36. A kit comprising:
a feeding tube, optionally according to any one of claims 24-35; and
a bacteria infusion device of any one of claims 1-23.

37. The kit of claim 36, wherein the bacteria infusion device further comprises an outlet fitting for coupling the outlet to the feeding tube.

38. A method of assembling the kit of claim 36 or 37, wherein the bacteria infusion device has a proximal end and a distal end, the method comprising coupling the distal end to the feeding tube.

39. The method of claim 38, further comprising coupling the proximal end to an enteral feed source.

40. A method of administering a non-pathogenic, intestinally beneficial bacteria to a patient having a feeding tube inserted into the gastrointestinal tract, the method comprising infusing a non-pathogenic, intestinally beneficial bacteria into the patient's gastrointestinal tract.

41. The method of claim 40, wherein the infusion of bacteria is performed through the feeding tube.

42. The method of claim 40 or 41, wherein the bacteria is in a liquid medium.

43. The method of claim 42, wherein the volume of liquid medium is in the range of 0.3 ml to 5 ml.

44. The method of any one of claims 40-43, further comprising, prior to use, storing the bacteria at a temperature that is below 20° C.

45. The method of any one of claims 40-44, further comprising repeating the step of infusing the bacteria into the gastrointestinal tract.

46. The method of claim 45, wherein continued infusion of bacteria is for the duration that enteral feeding is required.

47. The method of claim 45 or 46, wherein the repeated infusion is performed within 12 hours to 72 hours after the initial infusion.

48. The method of any one of claims 40-47, wherein the bacteria is infused simultaneously with infusion of an enteral feed.

49. The method of any one of claims 40-48, wherein the enteral feed comprises an oligosaccharide found in mammalian milk.

50. The method of any one of claims 40-49, further comprising coupling a bacteria infusion device of any one of claims 1-23 to the feeding tube, and wherein the bacteria and/or its supernatant is infused out from the internal chamber through the outlet.

51. The method of claim 50, further comprising coupling an enteral feed source to the bacteria infusion device.

52. The method of claim 51, further comprising infusing the enteral feed source through the bacteria infusion device and into the feeding tube.

53. The method of claim 51 or 52, further comprising detaching the bacteria infusion device after the enteral feed infusion is complete.

54. The method of any one of claims 40-53, wherein the feeding tube comprises:
an outer surface; and
a coating on the outer surface of the feeding tube, the coating comprising an antibacterial agent.

55. The method of any one of claims 40-54, wherein the patient is a preterm infant.
